(19) United States
(12) Patent Application Publication
(10) Pub. No.: US 2011/0039954 A1
(43) Pub. Date: Feb. 17, 2011

(54) CATALYST AND METHOD FOR SINGLE-STEP CONVERSION OF SYNGAS TO HYDROCARBON COMPOUNDS

(75) Inventors: Jianli Hu, Overland Park, KS (US); Jon C. Erickson, Overland Park, KS (US)

Correspondence Address: Hovey Williams LLP, 10801 Mastin Blvd., Suite 1000, Overland Park, KS, 66210, US

(73) Assignee: BLACK & VEATCH CORPORATION, Overland Park, KS (US)

(21) Appl. No.: 12/856,139

(22) Filed: Aug. 13, 2010

Related U.S. Application Data
(60) Provisional application No. 61/274,276, filed on Aug. 14, 2009.

(51) Int. Cl. C07C 1/04; B01J 23/26; B01J 23/60; B01J 29/06; B01J 8/02

(52) U.S. Cl. 518/709; 502/319; 502/339; 502/307; 502/329; 502/60; 502/77; 422/212; 422/617; 518/728

(57) ABSTRACT

Multi-functional catalyst and processes utilizing the catalyst in single-stage conversion of syngas into hydrocarbon compounds are provided. The multi-functional catalyst, which comprises two or more catalytic materials situated within molecular distances of each other, facilitates conversion of syngas into one or more intermediate compounds and then into desired hydrocarbon compounds, such as high octane gasoline, diesel, jet fuel, olefins, and xylenes.

### RELATED APPLICATION

The present application claims the benefit of U.S. Provisional Patent Application No. 61/274,276, entitled “Flexible Single Step Conversion of Syngas to Gasoline,” filed Aug. 14, 2009, which is incorporated by reference herein in its entirety.

### BACKGROUND OF THE INVENTION

1. Field of the Invention

The present invention generally pertains to a catalyst and process employing the catalyst in the direct conversion of a syngas mixture into hydrocarbon compounds, such as high octane gasoline, diesel, jet fuel, olefins, and xylenes, in a single stage. The catalyst generally comprises more than one catalytic material, each of the catalytic materials catalyzing one or more reactions in a particular reaction scheme.

2. Description of the Prior Art

As the price of oil climbs, the conversion of syngas into liquid hydrocarbon fuels attracts significant attention. Two processes have been demonstrated to make hydrocarbon fuels directly from syngas: (1) Fischer-Tropsch Synthesis (2) Exxon Mobil's Methanol to Gasoline (MTG) process. The Fischer-Tropsch (FT) process produces a wide range of paraffinic hydrocarbons with carbon numbers ranging from C1 to C90 or even higher. Without further upgrading, such as hydrocracking and isomerization, FT products can only be distilled to make diesel fuel.

The MTG process converts syngas to methanol and then to mainly a gasoline range hydrocarbon mixture containing C2-C10 hydrocarbon compounds. FIG. 1 schematically depicts the three-stage MTG process. The first stage converts a syngas mixture to methanol, typically employing a copper or zinc catalyst, at a reactor temperature of approximately 220° C. In the second reaction stage, the methanol is converted to dimethylether (DME) at a reactor temperature of approximately 300° C. In the third stage, the DME is converted to gasoline using a zeolite catalyst at a reaction temperature of approximately 400° C. The capital and operating costs involved with the MTG process can make this process quite unattractive from an economic viewpoint.

Haldor Topsoe has developed a modified version of the MTG process, known as TIGAS, which combines the methanol synthesis and DME synthesis stages in a single reactor. The TIGAS process is schematically depicted in FIG. 2. However, this process still presents similar drawbacks to the MTG process in that multiple reactors, operating at different temperatures, must be used. Thus, the TIGAS process is still very capital and operational cost intensive.

### SUMMARY OF THE INVENTION

In one embodiment according to the present invention, there is provided a multi-functional catalyst for use in the conversion of syngas into hydrocarbon compounds comprising at least first and second catalytic materials. The first catalytic material is generally functional to facilitate synthesis of one or more intermediate compounds from a syngas mixture. The second catalytic material is generally functional to facilitate synthesis of one or more hydrocarbon compounds from the one or more intermediate compounds. In some embodiments, one of the catalytic materials functions as a support for the other catalytic material. In those embodiments, often the first catalytic material is loaded upon the second catalytic material. However, it is within the scope of the present invention for the reverse to be the case as well.

In another embodiment according to the present invention, there is provided a system for converting syngas into hydrocarbon compounds comprising a reaction vessel containing a multi-functional catalyst as described herein. The system also includes a syngas feed stream for supplying syngas to the reaction vessel, and a product stream for removing the reaction products produced using the catalyst from the reaction vessel.

In yet another embodiment according to the present invention, there is provided a process for converting syngas into hydrocarbon compounds. Generally, the process comprises supplying a syngas mixture to a reaction vessel containing a multi-functional catalyst as described herein. The syngas is reacted in the presence of the catalyst to produce a hydrocarbon-containing product stream. In particular embodiments, the entire conversion of syngas into the desired hydrocarbon compounds occurs within a single reaction vessel, thereby eliminating the need for a plurality of serial reactors, each of which comprise different catalysts for performing different steps of the overall reaction scheme.

### BRIEF DESCRIPTION OF THE DRAWINGS

FIG. 1 is a schematic illustration of the prior art ExxonMobil MTG process;

FIG. 2 is a schematic illustration of the prior art Haldor Topsoe TIGAS process;

FIG. 3 is a schematic illustration of a single-stage process for producing gasoline from syngas in accordance with the present invention;

FIG. 4 is an exemplary catalyst made in accordance with the present invention;

FIG. 5 is another exemplary catalyst made in accordance with the present invention;

FIG. 6 is a schematic, cross-sectional view of a catalytic membrane reactor that may be used in certain embodiments according to the present invention; and

FIG. 7 is a schematic representation of a moving-bed reactor that may be used in certain embodiments according to the present invention.

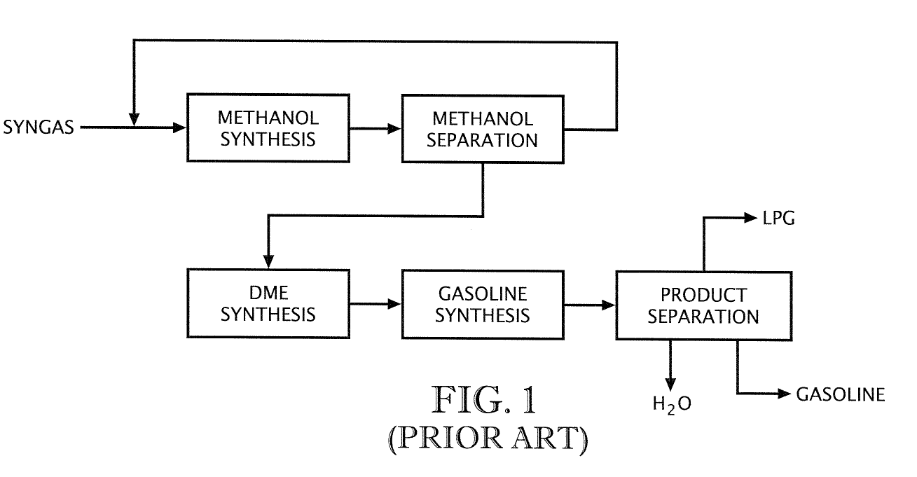

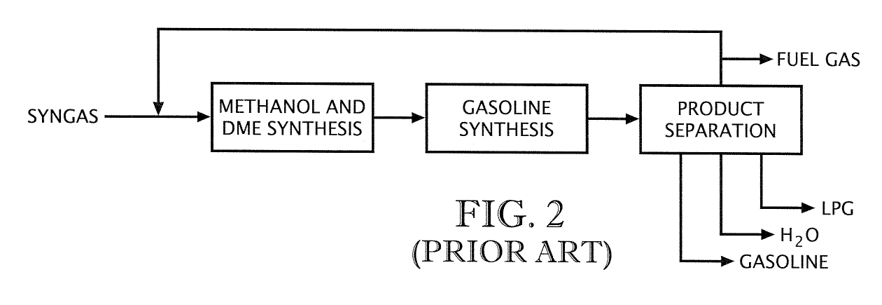

### DETAILED DESCRIPTION OF THE PREFERRED EMBODIMENT

Synthesis gas, or syngas, generally refers to a mixture comprising carbon monoxide and hydrogen, and very often some carbon dioxide. Syngas may be produced from any carbonaceous feedstock according to various processes. For example, syngas may be produced by steam reforming of natural gas or liquid hydrocarbons, or through the gasification of biomass, lignin, coal, or petcoke.

The present invention exhibits great flexibility in the type of products that may be produced. In one embodiment, the present invention may be directed toward the production of hydrocarbon compounds, and particularly C5-C10 hydrocarbon compounds which generally comprise gasoline. The process may also be utilized to produce a number of other carbon-containing compounds such as olefins, aromatics, alcohols such as ethanol, ethers such as DME, and other chemical intermediates.

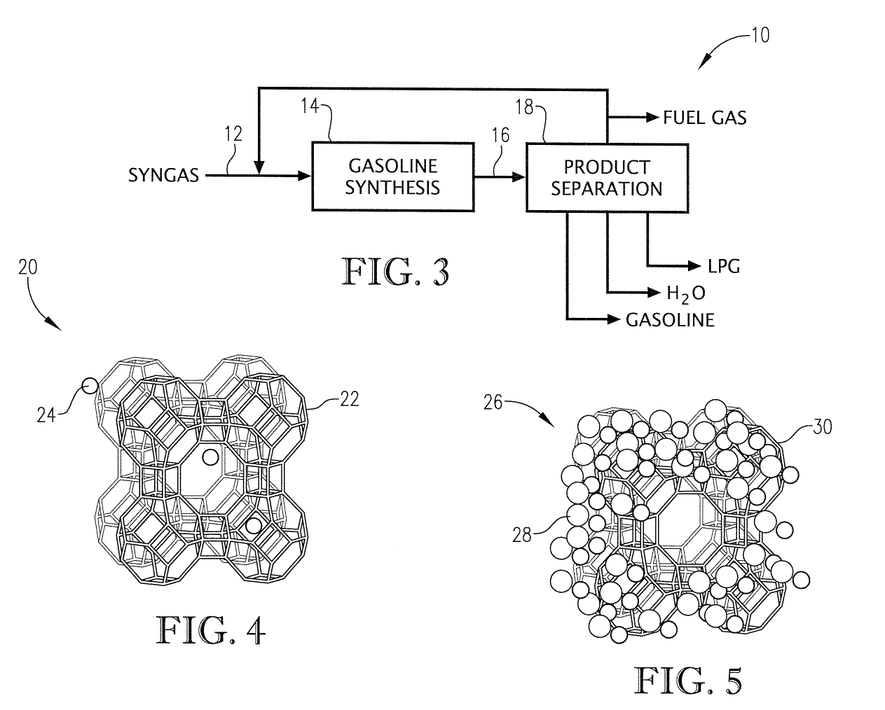

Turning first to FIG. 3, a process 10 according to one embodiment of the present invention is illustrated. In process 10, a syngas feed stream 12 is directed to a reactor 14, in which resides a quantity of a catalyst, which is described in greater detail hereafter. Within reactor 14, direct conversion of the syngas mixture into a desired final product is achieved. A product stream 16 carries the reaction products created using the catalyst from reactor 14 toward, if necessary, a separation system 18 where the reaction products and/or unreacted portions of the feed components are separated from each other. These reaction products include gasoline grade hydrocarbons, water, and liquified petroleum gas (LPG). Unreacted syngas components can be recycled to feed stream 12 or used as fuel gas.

In the foregoing example where gasoline is a desired product, the reaction mechanism taking place within the reactor may be as follows:

2H₂+CO→CH₃OH

2CH₃OH→CH₃OCH₃+H₂O

CH₃OCH₃→[CH₂−CH₂]+H₂O

with [CH₂−CH₂] being an average representation of the hydrocarbon product. In this process, two intermediate compounds are produced, methanol and dimethylether. As explained in greater detail below, given the close proximity of the intermediate products upon formation to the various catalytic materials contained within the catalyst, high yields of the hydrocarbon product can be achieved in a single reactor vessel.

Catalysts according to the present invention are multi-functional given that they are capable of catalyzing more than one chemical reaction. In certain embodiments, and particularly with respect to the process described above for producing gasoline, the catalysts are at least tri-functional. Generally, the catalyst comprises at least first and second catalytic materials, although a plurality of catalytic materials may be present within the catalyst. In certain embodiments, one of the catalytic materials may function as a support for one or more other catalytic materials which can be loaded thereon. The close proximity of the various catalytic materials to each other assists with the ready conversion of a reaction product produced with the first catalytic material to a reaction product produced with the second catalytic material, and so forth.

The catalyst may be in the form of discrete particles that can be loaded into a reactor vessel. However, it is within the scope of the present invention for the catalyst to be deposited on some other type of support material, such as a ceramic honeycomb. In those embodiments in which one of the catalytic materials functions as a support, the supporting catalytic material generally presents a surface area of at least 200 m²/g, or between about 200 m²/g to about 800 m²/g, or between about 300 m²/g to about 700 m²/g. The supporting catalytic material may also have a pore volume of at least 0.5 cm³/g, or between about 0.5 cm³/g to about 2.5 cm³/g, or between about 0.75 cm³/g to about 1.5 cm³/g. In certain embodiments, the supporting catalytic material also presents pore opening sizes of between about 2 Å to about 10 Å, or between about 4 Å to about 8 Å, or between about 5 Å to about 6.5 Å. In particular embodiments, such as shown in FIGS. 4 and 5, the supporting catalytic material has a crystalline structure which presents a central pore or channel through the material. This channel generally presents a length of between about 10 Å to about 60 Å, or between about 20 Å to about 40 Å, or between about 25 Å to about 35 Å, or about 30 Å. In one embodiment according to the present invention, the supporting catalytic material comprises a zeolite, such as ZSM-5. The zeolite may have a ratio of silicon to aluminum of between about 20 to about 40, or between about 25 to about 35.

The catalyst according to the present invention can be produced according to various techniques including ion-exchange and precipitation processes. The catalyst 20 depicted in FIG. 4 has been prepared by an ion-exchange technique. A supporting catalytic material 22, such as zeolite, generally comprises protons attached to its external and internal (i.e., pore) surfaces. In the ion-exchange technique, these protons are replaced by another catalytic material 24, which generally comprises one or more metal species. Thus, the catalytic material which replaces the protons strongly interacts, or bonds, with the supporting catalytic material, and a close proximity between the two catalytic materials is maintained.

In an exemplary embodiment, the catalytic material which is loaded upon the supporting catalytic material is capable of converting syngas into methanol. In certain embodiments, this catalytic material comprises palladium or chromium, and in even further embodiments, may also include a dehydration catalyst such as a metal oxide, and more particularly, zinc oxide. The methanol catalyst may be provided as a salt, such as PdCl₂. The palladium cation is then exchanged for a proton located on an exposed surface of the zeolite.

The catalyst 26, depicted in FIG. 5, has been prepared by a precipitation technique. In this technique, a catalytic material 28 is loaded upon a supporting catalytic material 30. In this embodiment, catalytic material 28 is loaded onto the external surfaces of catalytic material 30. In certain embodiments, this loading of catalytic material 28 at least partially coats the external surfaces of catalytic material 30. In still other embodiments, catalytic material 28 encapsulates catalytic material 30. Loading of catalytic material 28 is such that the internal pores of catalytic material 30 are not blocked, as it remains important for the reaction products produced using catalytic material 28 to have immediate and unobstructed access to catalytic material 30.

In an exemplary embodiment, catalytic material 28, much like catalytic material 24 from FIG. 4, is functional to convert syngas into methanol. Initially, catalytic material 28 may be provided as a salt solution which is then mixed with a quantity of zeolite. In particular embodiments, the salt comprises palladium and/or chromium, and the solvent used to solubilize the salt comprises an aqueous ammonia solution (i.e., ammonium hydroxide). The solvent is driven off and the remaining salt becomes deposited on the zeolite.

In another embodiment according to the present invention, the catalyst comprises a first catalytic material loaded upon a second catalytic material. The first catalytic material is functional to facilitate synthesis of one or more intermediate compounds from a syngas mixture. In certain embodiments, the first catalytic material is functional to facilitate synthesis of methanol and/or dimethylether (DME) from the syngas mixture. In still other embodiments, the first catalytic material comprises palladium or chromium, and in even further embodiments, the first catalytic material comprises Pd/ZnO or Cr/ZnO. The second catalytic material is functional to facilitate synthesis of one or more hydrocarbon compounds from the one or more intermediate compounds. In certain embodiments, the second catalytic material comprises a zeolite, and particularly ZSM-5. These catalysts are particularly well suited for single-step conversion of syngas into gasoline-grade hydrocarbon compounds.

In the production of gasoline-grade hydrocarbon compounds, the catalyst is utilized at reaction temperatures of between about 350 to about 400° C. As described above, at least a portion of the syngas mixture is first converted to methanol through the action of the methanol catalyst deposited on the zeolite. The metal oxide portion of the first catalytic material facilitates dehydration of the methanol thereby forming DME. Given the nature of the catalyst, the synthesis of DME occurs in close proximity to the zeolite portion of the catalyst. Thus, upon its formation, DME easily migrates into the channel portion of the zeolite crystal structure which facilitates the conversion of DME into gasoline grade hydrocarbons, predominantly C5-C10 hydrocarbon compounds. In this reaction scheme, the methanol synthesis rate is much faster than the subsequent methanol to gasoline formation rate. Therefore, the overall conversion of syngas to gasoline is limited by the diffusion of methanol within the zeolite. Use of medium porous zeolite and shorter pore length (small crystal) zeolite may improve the diffusion characteristics of the catalyst.

In certain embodiments, the present invention can be used to synthesize in a single reaction step a hydrocarbon mixture that is compatible with existing petroleum-derived gasoline infrastructure. Table 1, below, compares the common properties of petroleum-derived gasoline and gasoline produced according to a methanol-to-gasoline process.

TABLE 1

| | Petroleum Derived Gasoline | MTG Gasoline |
|---|---|---|
| Oxygen (wt %) | 1.08 | 0 |
| API Gravity | 61.9 | 61.8 |
| Aromatics (vol %) | 24.7 | 26.5 |
| Olefins (vol %) | 11.6 | 12.6 |
| Reid Vapor Pressure | 12.12 | 9 |
| (RAP) | | |
| T50 (° F.) | 199.9 | 201 |
| T90 (° F.) | 324.1 | 320 |
| Sulfur (ppm) | 97 | 0 |
| Benzene (vol %) | 1.15 | 0.3 |

In addition to producing gasoline products, the catalyst can be tuned adjust the type and quantity of hydrocarbon compounds produced. Typically, this tuning of the catalyst involves adjusting how much of the first catalytic material is loaded onto the second catalytic material. Also, selection of different catalytic materials, either chemically different or having different physical characteristics may affect the composition of the final hydrocarbon product. For example, the catalyst can be tuned so as to favor formation of p-xylene, a starting material for the production of polyesters.

The reaction products of the methanol-to-gasoline reaction scheme generally comprise a hydrocarbon portion which itself predominantly comprises C5-C10 hydrocarbon compounds. However, the reaction scheme also results in the production of significant quantities of water, approximately 58 wt. % of the total reaction products. Under the reaction conditions employed in the present MTG process (350-400° C., at several atmospheres of pressure), this water is in the form of high-pressure steam. The presence of high concentrations of steam reduces the H₂ and CO partial pressures, which can result in a decreased reaction rate along the length of the reactor. Furthermore, high temperature steam can cause irreversible deactivation of the zeolite catalytic material due to the breakdown of its crystal structure. While any type of reactor vessels may be used with the present invention, certain reactor types present advantages in view of these issues.

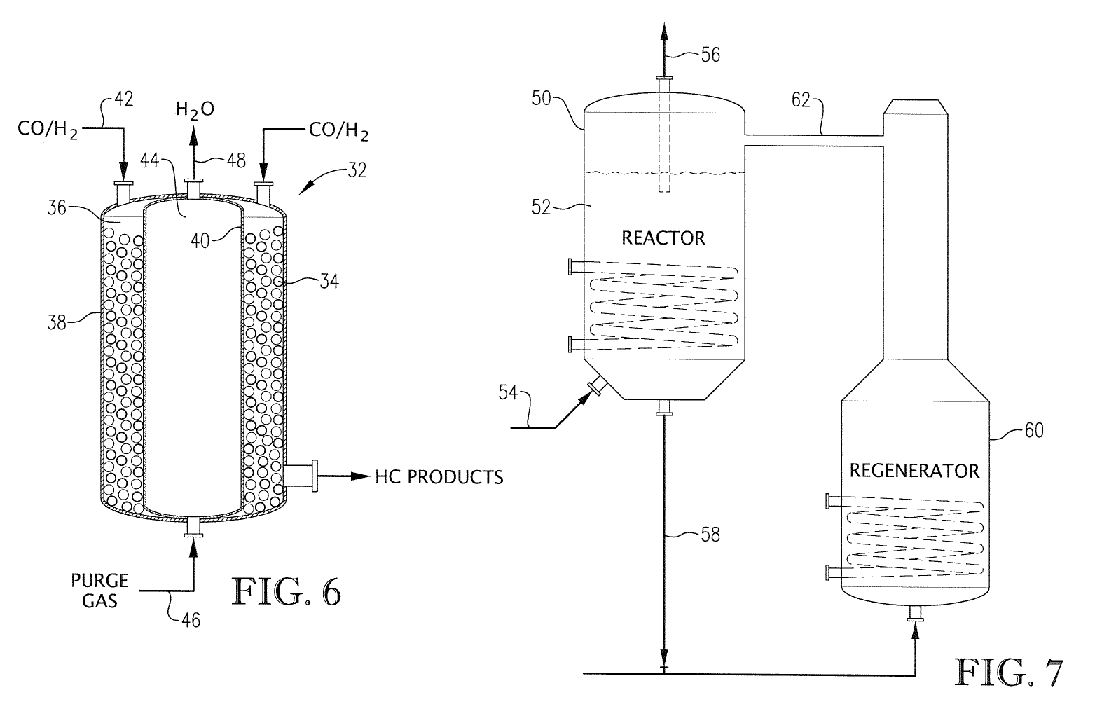

In one embodiment of the present invention, a catalytic membrane reactor is used so that steam generated during the MTG reaction process can be removed from the reactor “in-situ.” FIG. 6 illustrates an exemplary catalytic membrane reactor 32. In one particular embodiment of this type of reactor, catalyst 34 is loaded into an annular chamber 36 defined by the outer wall 38 of reactor 32 and an inner, cylindrical, hydrophilic membrane 40. Membrane 40 may be formed of a hydrophilic zeolite, such as sodalite, and selectively permits passage of water therethrough, and substantially rejects other reaction products produced within chamber 36. The syngas mixture 42 is fed into annular chamber 36 where the reaction proceeds in the presence of catalyst 34. The pressure within annular chamber 36 drives the produced steam through membrane 40 into central passage or chamber 44. An inert purge gas stream 46 is directed through passage 44 thereby directing the steam out of reactor 32 via stream 48. Thus by purging the water from reactor 32, deactivation of catalyst 34 is slowed.

In another embodiment of the present invention, a moving-bed reactor can be utilized to facilitate withdrawal of spent catalyst from the reactor and addition of fresh catalyst into the reactor. FIG. 7 illustrates an exemplary moving-bed reactor 50 that contains a catalyst 52. The syngas reactants are delivered to reactor 50 via stream 54 where they react in the presence of catalyst 52 to produce a stream of reaction products, which are removed from reactor via stream 56. Spent catalyst 52 can be withdrawn from reactor 50 via stream 58 and sent to a catalyst regenerator 60. Regenerated catalyst can be introduced into reactor 50 as needed via conduit 62. It is also recognized that fresh catalyst may be introduced into reactor 50 via conduit 62 in addition to or in place of regenerated catalyst as needed.

Moving-bed reactor 50 also readily permits modification of the reaction product distribution without having to stop reactor operation to replace the catalyst. As discussed above, the catalyst to be used with the present invention can be tuned so as to provide reaction products comprising desired levels of specific hydrocarbon compounds. For example, if it was desired to produce significant middle distillate, diesel range hydrocarbons, the catalyst composition within reactor 50 could be modified without taking the reactor off-line. Thus, the mixture of catalyst within reactor 50 could be varied over time to allow the ratio of gasoline to diesel and jet fuel to be adjusted according to seasonal market demands.

It is understood that the embodiments of the present invention described herein are provided by way illustration and nothing therein should be taken as limiting the scope of the invention.

We claim:

1. A multi-functional catalyst for use in the production of hydrocarbon compounds comprising:
a first catalytic material functional to facilitate synthesis of one or more intermediate compounds from a syngas mixture; and
a second catalytic material functional to facilitate synthesis of one or more hydrocarbon compounds from said one or more intermediate compounds.

2. The multi-functional catalyst according to claim 1, wherein said second catalytic material functions as a support for said first catalytic material, said first catalytic material being loaded upon said second catalytic material.

3. The multi-functional catalyst according to claim 2, wherein said first catalytic material is deposited onto at least a portion of the outer surface of said second catalytic material.

4. The multi-functional catalyst according to claim 2, wherein said first catalytic material comprises a metal species that is chemically bonded to the outer surfaces and/or pore surfaces of said second catalytic material.

5. The multi-functional catalyst according to claim 4, wherein said metal species is chemically bonded to said second catalytic material through an ion-exchange process.

6. The multi-functional catalyst according to claim 1, wherein said one or more intermediate compounds comprise methanol and/or dimethylether.

7. The multi-functional catalyst according to claim 1, wherein said first catalytic material comprises palladium or chromium.

8. The multi-functional catalyst according to claim 7, wherein said first catalytic material comprises Pd/ZnO or Cr/ZnO.

9. The multi-functional catalyst according to claim 1, wherein said second catalytic material comprises a zeolite.

10. The multi-functional catalyst according to claim 9, wherein said zeolite is ZSM-5.

11. The multi-functional catalyst according to claim 9, wherein said zeolite has a pore length of between about 20 to about 40 Å.

12. The multi-functional catalyst according to claim 9, wherein said zeolite has a pore opening of between about 5 to about 6.5 Å.

13. The multi-functional catalyst according to claim 9, wherein said zeolite has a surface area of at least 200 m²/g.

14. The multi-functional catalyst according to claim 9, wherein said zeolite has a pore volume of at least 0.5 cm³/g.

15. The multi-functional catalyst according to claim 1, wherein said catalyst is operable to facilitate conversion of the syngas mixture into hydrocarbon compounds at processing conditions of between about 350 to about 400° C.

16. A system for converting syngas into hydrocarbon compounds comprising:
a reaction vessel containing the multi-functional catalyst according to claim 1;
a syngas feed stream for supplying syngas to said reaction vessel; and
a product stream for removing reaction products produced using said catalyst from said reaction vessel.

17. The system according to claim 16, wherein said reaction vessel comprises a catalytic membrane reactor operable to separate at least a portion of the water being generated therein by a reaction process used to synthesize the hydrocarbon compounds.

18. The system according to claim 16, wherein said reaction vessel comprises a moving-bed reactor, from which catalyst may be withdrawn and/or added without stopping the reaction occurring within the reactor vessel.

19. A process for producing hydrocarbon compounds comprising the steps of:
supplying a syngas mixture to a reaction vessel containing the multi-functional catalyst according to claim 1; and
reacting said syngas mixture within said reaction vessel to produce a hydrocarbon-containing product stream.

20. The process according to claim 19, wherein the hydrocarbon portion of said product stream predominantly comprises C5-C10 hydrocarbon compounds.

21. The process according to claim 19, wherein said reacting step is carried out at a temperature of between about 350 to about 400° C.

22. The process according to claim 19, wherein said reaction vessel comprises a catalyst chamber and a water-removal chamber, said chambers being separated by a hydrophilic membrane which selectively permits passage of water therethrough.

23. The process according to claim 22, wherein said reaction which produces said hydrocarbon-containing product stream also produces water, said water being present within said reaction vessel as steam, at least a portion of said steam passing through said hydrophilic membrane into said water-removal chamber, and wherein said process further comprises passing a purge gas through said water-removal chamber so as to remove at least a portion of said steam within said water-removal chamber from said reaction vessel.

24. The process according to claim 19, wherein said reaction vessel comprises a moving-bed reactor.

25. The process according to claim 24, said method further comprising removing catalyst from said reactor and directing said removed catalyst toward a catalyst regeneration system.

26. The process according to claim 25, said process further comprising adding catalyst to said reactor, said added catalyst being fresh catalyst or regenerated catalyst from said regeneration system.

27. The process according to claim 25, said added catalyst having functional characteristics that are different from the catalyst being removed so that the composition of said product stream is altered.
